(19) United States
(12) Patent Application Publication
(10) Pub. No.: US 2017/0115219 A1
(43) Pub. Date: Apr. 27, 2017

(54) GAS ANALYZING APPARATUS

(71) Applicant: HORIBA, Ltd., Kyoto-shi (JP)

(72) Inventor: Takuya Ido, Kyoto-shi (JP)

(21) Appl. No.: 15/332,961

(22) Filed: Oct. 24, 2016

(30) Foreign Application Priority Data
Oct. 26, 2015 (JP) 2015-209909

(51) Int. Cl. G01N 21/59; G01N 33/00

(52) U.S. Cl. CPC G01N 21/59; G01N 33/0073; G01N 2201/12; G01N 2201/0612

(57) ABSTRACT

A gas analyzing apparatus includes a plurality of light sources, an inlet, a light detector, and an analyzing unit. The plurality of light sources simultaneously output a plurality of measurement light beams. The inlet introduces the plurality of measurement light beams into a measurement space. The light detector measures total intensity. The analyzing unit analyzes the target gases based on a difference between a measured target intensity and a reference intensity, in which the measured target intensity is a total intensity measured by the light detector after passing through the measurement space in which one of the target gases exists, while the reference intensity is the total intensity measured by the light detector after passing through the measurement space in which none of the target gases exists.

### CROSS-REFERENCE TO RELATED APPLICATIONS

This application claims priority under 35 U.S.C. §119 to Japanese Patent Applications No. 2015-209909 filed on Oct. 26, 2015. The entire disclosure of Japanese Patent Applications No. 2015-209909 is hereby incorporated herein by reference.

### BACKGROUND OF THE INVENTION

Field of the Invention

The present invention relates to a gas analyzing apparatus for analyzing a target gas that exists in a measurement space.

Description of the Related Art

Conventionally, there is known an apparatus for analyzing a target gas existing in a measurement space based on a light absorption amount of a measurement light beam after passing through the measurement space.

For example, JP-A-11-142327 discloses an apparatus that introduces into a gas atmosphere a measurement light beam, which is a combination of a laser beam having a wavelength that is absorbed by the target gas and a laser beam having a wavelength that is not absorbed by the target gas, and extracts an intensity of the laser beam of a wavelength that is absorbed by the target gas and the intensity of the laser beam of a wavelength that is not absorbed by the target gas from the measurement light beam received by a light detector after passing through the gas atmosphere. In JP-A-11-142327, the target gas is analyzed based on a difference between the intensity of the laser beam of a wavelength that is absorbed by the target gas and the intensity of the laser beam of a wavelength that is not absorbed by the target gas.

As described above, in order to accurately analyze the target gas using light absorption by the target gas, a light beam (a reference light beam) is necessary as a reference of the light absorption amount by the target gas. Therefore the apparatus of JP-A-11-142327 includes a light source for generating the light beam (the measurement light beam) to be absorbed by the target gas and a light source for generating the reference light beam, separately. In the case of the conventional apparatus including the light source for the measurement light beam and the light source for the reference light beam separately, two light sources are necessary for each gas when measuring plural gases by one apparatus. In other words, the conventional apparatus needs light sources of twice the number of gases to be measured. In addition, the conventional apparatus cannot accurately analyze the target gas when analyzing based on a difference between the intensity of the reference light beam and the intensity of the measurement light. It is because a deviation of characteristics occurs between the light sources disposed separately and varies for each measurement.

### SUMMARY OF THE INVENTION

It is an object of the present invention to accurately analyze a target gas with an apparatus for analyzing a target gas using light absorption by the target gas.

A plurality of aspects of the present invention achieving the object are described below. These aspects can be arbitrarily combined as necessary.

A gas analyzing apparatus according to one aspect of the present invention analyzes gases existing in a measurement space. The gas analyzing apparatus includes a plurality of light sources, an inlet, a light detector, and an analyzing unit.

The plurality of light sources simultaneously output a plurality of measurement light beams of unique wavelength ranges, each of the plurality of measurement light beams is capable of being absorbed by one of a plurality of target gases that exists in the measurement space. The inlet introduces the plurality of measurement light beams output simultaneously from the plurality of light sources into the measurement space. The light detector measures a total intensity obtained by summing intensities of the plurality of measurement light beams that have been introduced from the inlet and passed through the measurement space.

The analyzing unit analyzes the plurality of target gases based on a difference between a measured target intensity and a reference intensity. The measured target intensity is the total intensity measured by the light detector when the plurality of measurement light beams pass through the measurement space in which one of the plurality of target gases exists, while the reference intensity is the total intensity measured by the light detector when the plurality of measurement light beams pass through the measurement space in which none of the plurality of target gases exists.

In this way, the gas analyzing apparatus can accurately analyze the plurality of target gases without disposing light sources respectively to the target gases to measure the reference intensity.

The inlet may include an optical multiplexer. The optical multiplexer generates a multiplexed measurement light beam by multiplexing the plurality of measurement light beams output simultaneously from the plurality of light sources to introduce the multiplexed measurement light beam into the measurement space. The multiplexed measurement light has an intensity. In this case, the light detector measures intensity of the multiplexed measurement light beam as the total intensity.

In this way, the plurality of measurement light beams output simultaneously from the plurality of light sources can be efficiently introduced into the measurement space.

The inlet may further include a light splitter. The light splitter divides the multiplexed measurement light beam to output the multiplexed measurement light beam from a plurality of light outlets. In this way, the multiplexed measurement light beam can be output to a space other than the measurement space.

The inlet may introduce the plurality of multiplexed measurement light beams output from the plurality of light outlets respectively into a plurality of measurement spaces. In this way, it is not necessary to dispose light sources respectively for the measurement spaces to analyze the target gases in the plurality of measurement spaces.

The gas analyzing apparatus may further include a gas type reporting unit that reports which target gas exists in the measurement space. In this way, even if a type of the target gas that exists in the measurement space cannot be determined from the measured target intensity, it is possible to know which target gas exists in the measurement space.

Each of the plurality of light sources may be configured to increase or decrease the wavelength of the measurement light beam within the unique wavelength range at a predetermined period. In this way, it is possible to generate the multiplexed measurement light beam whose wavelength temporally changes.

A periodic increase/decrease pattern of some of a plurality of drive currents input to some of the plurality of light sources may be different from periodic increase/decrease patterns of other drive currents. In this way, a dynamic range in analyzing the target gases can be increased.

The analyzing unit may analyze the target gases based on a difference between a temporal change of the measured target intensity and a temporal change of the reference intensity. In this way, even if influence of the light absorption by the target gas to the measured target intensity is small, the target gas can be analyzed more accurately.

A gas analyzing apparatus according to another aspect of the present invention is a gas analyzing apparatus that analyzes a target gas existing in a measurement space. The gas analyzing apparatus includes a light source, an inlet, a light detector, and an analyzing unit.

The light source outputs a measurement light beam having a unique wavelength range. The measurement light beam is capable of being absorbed by the target gas. The inlet introduces the measurement light beam output from the light source into the measurement space. The light detector measures an intensity of the measurement light beam that has been introduced from the inlet and passed through the measurement space.

The analyzing unit analyzes the target gas in the measurement space in which the target gas exists at a concentration lower than a first concentration, based on a difference between a reference intensity and a measured target intensity. The reference intensity is the intensity measured by the light detector when the measurement light beam passes through the measurement space in which the target gas exists at the first concentration. The measured target intensity is the intensity measured by the light detector when the measurement light beam passes through the measurement space in which the target gas exists at a concentration lower than the first concentration.

In this way, the target gas in the measurement space in which the target gas exists at a low concentration can be accurately analyzed.

Thus, it is possible to accurately analyze the target gas in the apparatus that analyzes the target gas using light absorption by the target gas.

### BRIEF DESCRIPTION OF THE DRAWINGS

FIG. 1 is a diagram illustrating a structure of a gas analyzing apparatus.

FIG. 2 is a diagram illustrating a structure of a controller.

FIG. 3 is a diagram illustrating an example of drive currents output to light sources in a first embodiment.

FIG. 4 is a flowchart illustrating an operation of a gas analyzing apparatus according to the first embodiment.

FIG. 5 is a diagram illustrating an example of profiles of measurement light beams and total intensity in cases where respective gases exist.

FIG. 6 is a diagram schematically illustrating a method of calculating analysis data.

FIG. 7 is a diagram illustrating an example of calculating a “peak to bottom” value from the analysis data.

FIG. 8 is a diagram illustrating a structure of a gas analyzing apparatus according to a second embodiment.

FIG. 9 is a diagram illustrating a structure of a gas analyzing apparatus as a variation of the second embodiment.

FIG. 10A is a diagram illustrating an example of the drive currents output to the light sources in a third embodiment.

FIG. 10B is a diagram illustrating another example of the drive currents output to the light sources in the third embodiment.

FIG. 11 is a diagram illustrating an example of gas type dependency of the profile of the analysis data obtained when a pattern of periodical change of the plurality of measurement light beams is different.

FIG. 12 is a diagram illustrating an overall structure of a gas analyzing apparatus according to a fourth embodiment.

FIG. 13 is a diagram illustrating a structure of a controller according to the fourth embodiment.

FIG. 14 is a flowchart illustrating an operation of the gas analyzing apparatus according to the fourth embodiment.

FIG. 15 is a diagram illustrating an example of a temporal variation of concentration of target gas.

FIG. 16 is a diagram schematically illustrating a method of generating the analysis data.

FIG. 17A is a diagram illustrating an example of the analysis data obtained by subtracting a measured target intensity from a second reference intensity.

FIG. 17B is a diagram illustrating another example of the analysis data obtained by subtracting the measured target intensity from the second reference intensity.

### DETAILED DESCRIPTION OF THE INVENTION

### (1) First Embodiment

(1-1) Overall Structure of Gas Analyzing Apparatus

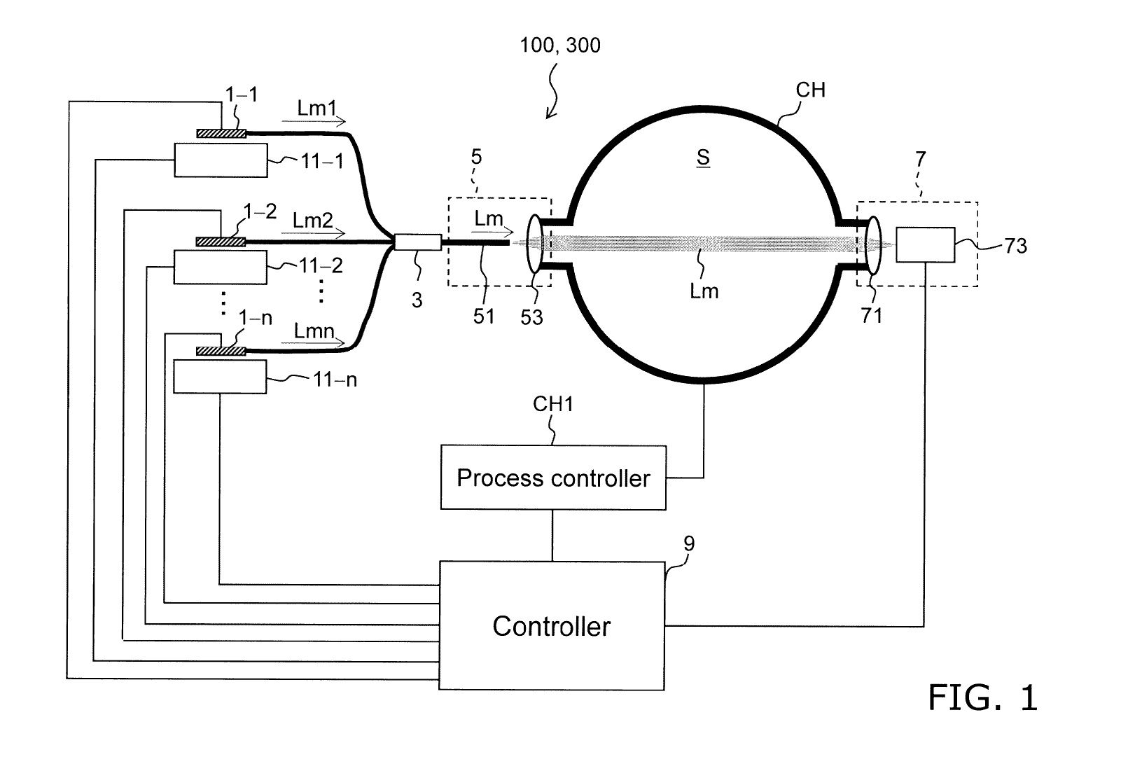

An overall structure of a gas analyzing apparatus 100 according to a first embodiment is described with reference to FIG. 1. FIG. 1 is a diagram illustrating a structure of the gas analyzing apparatus. The gas analyzing apparatus 100 is an apparatus that analyzes target gases that possibly exist in a measurement space S. In this embodiment, the measurement space S is an inner space of a chamber CH (FIG. 1) in which various surface treatment processes of solid materials are performed.

The gas analyzing apparatus 100 includes a plurality of light sources 1-1, 1-2, . . . , 1-n. The plurality of light sources 1-1, 1-2, . . . , 1-n respectively output measurement light beams Lm1, Lm2, . . . Lmn having unique wavelength ranges, each of which is absorbed by each target gas. The light source is a laser diode (LD), for example.

When analyzing target gases in the measurement space S, the plurality of light sources 1-1, 1-2, . . . , 1-n simultaneously output the plurality of measurement light beams Lm1, Lm2, . . . , Lmn.

Note that the number of the light sources provided to the gas analyzing apparatus 100 is not limited to be the same as the number of the target gases. For example, if a single light source can generate the measurement light beams that can be absorbed by a plurality of target gases, the number of the light sources can be smaller than the number of the target gases.

In addition, for example, when analyzing target gases of the number smaller than the light sources 1-1, 1-2, . . . , 1-n provided to the gas analyzing apparatus 100, some of the plurality of light sources 1-1, 1-2, . . . , 1-n may be selected to operate them simultaneously.

The plurality of light sources 1-1, 1-2, . . . , 1-n are equipped with temperature controlling apparatuses 11-1, 11-2, . . . , 11-n, respectively. Each of the temperature controlling apparatuses 11-1, 11-2, . . . , 11-n can include a Peltier device and a temperature sensor (e.g. a thermistor), for example.

The temperature controlling apparatuses 11-1, 11-2, . . . , 11-n respectively adjust temperatures of the plurality of light sources 1-1, 1-2, . . . , 1-n so that each of the plurality of light sources 1-1, 1-2, . . . , 1-n has a constant temperature. In this way, each of the plurality of light sources 1-1, 1-2, . . . , 1-3 can control wavelengths of the measurement light beams Lm1, Lm2, . . . , Lmn based on input current levels without being affected by the surrounding or ambient temperature.

The gas analyzing apparatus 100 includes an optical multiplexer 3. The optical multiplexer 3 multiplexes the plurality of measurement light beams Lm1, Lm2, . . . , Lmn output simultaneously from the plurality of light sources 1-1, 1-2, . . . , 1-n so as to generate a multiplexed measurement light beam Lm. The optical multiplexer 3 is a fiber coupler, for example. In this case, the optical multiplexer 3 multiplexes the measurement light beams Lm1, Lm2, . . . , Lmn transmitted from a plurality of optical fibers at a position where the plurality of optical fibers are fused, so as to generate the multiplexed measurement light beam Lm. The optical multiplexer 3 can also be an optical switch or the like.

The gas analyzing apparatus 100 is equipped with an inlet 5. The inlet 5 introduces the multiplexed measurement light beam Lm (i.e. the plurality of measurement light beams Lm1, Lm2, . . . , Lmn output simultaneously from the plurality of light sources 1-1, 1-2, . . . , 1-n) into the measurement space S. The inlet 5 includes an optical fiber 51. One end of the optical fiber 51 is connected to an outlet of the multiplexed measurement light beam Lm of the optical multiplexer 3. On the other hand, the other end of the optical fiber 51 is disposed in the vicinity of a collimator 53 (described later) disposed in an opening of a side wall of the chamber CH.

The inlet 5 includes the collimator 53. The collimator 53 converts the multiplexed measurement light beam Lm transmitted through the optical fiber 51 into a collimated light beam and introduces the same into the measurement space S.

The gas analyzing apparatus 100 includes a light detector 7. The light detector 7 measures a total intensity Im obtained by summing intensities of the plurality of measurement light beams Lm1, Lm2, . . . , Lmn that have been introduced from the inlet 5 and passed through the measurement space S. Specifically, the light detector 7 measures intensity of the multiplexed measurement light beam Lm that has passed through the measurement space S as the total intensity Im.

The light detector 7 includes a light collector 71. The light collector 71 is disposed at an opening that is opposed to the opening to which the collimator 53 is attached. The light collector 71 is a lens for condensing the multiplexed measurement light beam Lm that has been introduced from the inlet 5 and passed through the measurement space S onto a light receiving surface of a light receiving device 73 (described later).

The light detector 7 includes the light receiving device 73. The light receiving device 73 is a photodiode that generates an electric signal based on the intensity of the multiplexed measurement light beam Lm received by the light receiving surface. Alternatively, the light receiving device 73 may be a photomultiplier.

Note that the plurality of measurement light beams Lm1, Lm2, . . . , Lmn (the multiplexed measurement light beam Lm) introduced into the measurement space S may be received by the light detector 7 after being reflected (a plurality of times) in the measurement space S by a (plurality of) reflector(s) (not shown) disposed in the measurement space S and/or an inner wall of the chamber CH.

As the plurality of measurement light beams Lm1, Lm2, . . . , Lmn are reflected (a plurality of times) in the measurement space S, the length of the optical path of the plurality of measurement light beams Lm1, Lm2, . . . , Lmn in the measurement space S can be longer than the diameter of the chamber CH, for example.

The gas analyzing apparatus 100 includes a controller 9. The controller 9 is a computer system including a CPU, a storage device (a device that stores data such as a RAM and a ROM or the like), and various interfaces (such as an A/D converter, a D/A converter, and a communication circuitry). The controller 9 controls the elements of the gas analyzing apparatus 100. The controller 9 performs various information processes to analyze the target gases that exist in the measurement space S.

A structure of the controller 9 and information processes performed by the controller 9 will be described later.

The controller 9 may be connected to a process controller CH1 that includes a flow rate controller that controls an introducing amount of the target gas to be introduced into the measurement space S and/or a substance that generates the target gas, as illustrated in FIG. 1, for example.

In this case, by receiving a signal input to the flow rate controller, for example, the controller 9 can recognize which target gas exists in the measurement space S.

With the structure described above, the gas analyzing apparatus 100 can analyze the target gases in the measurement space S based on the total intensity Im of the multiplexed measurement light beam Lm received by the light detector 7 after passing through the measurement space S in the chamber CH.

(1-2) Structure of Controller

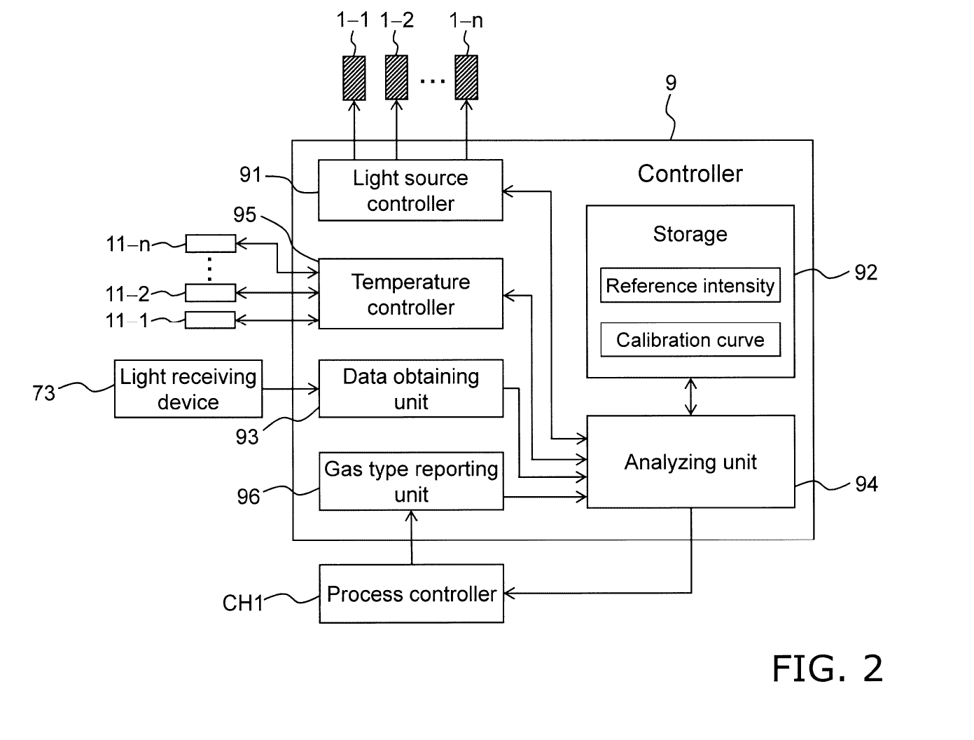

Next, a structure of the controller 9 is described with reference to FIG. 2. FIG. 2 is a diagram illustrating a structure of the controller. Note that a part of the functions of individual elements of the controller 9 described below may be realized by a program that is stored in the storage device and is executed by the computer system constituting the controller 9. A part of the functions of individual elements of the controller 9 may be realized by a custom integrated circuit (IC).

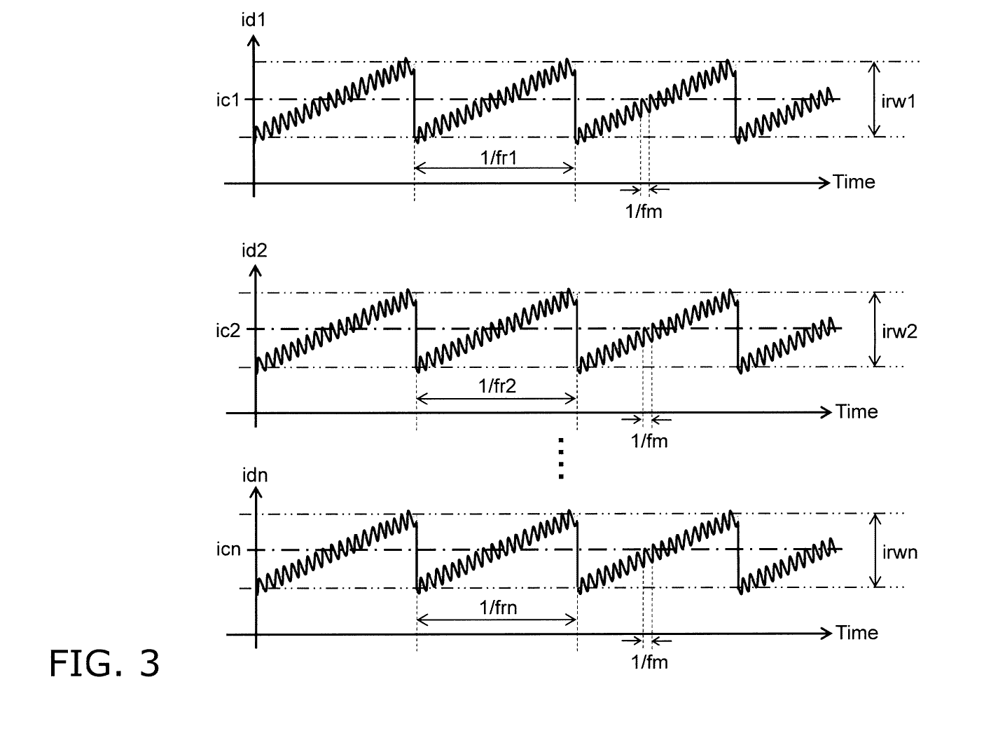

The controller 9 includes a light source controller 91. As illustrated in FIG. 3, the light source controller 91 sums each of mean currents ic1, ic2, . . . , icn that determines a center wavelength of the wavelength range of the measurement light beam output from each light source, each of ramp wave currents ir1, ir2, . . . , irn having amplitudes irw1, irw2, . . . , irwn and frequencies fr1, fr2, . . . , frn, and a modulation current imod that is a sine wave having a frequency fm higher than the frequencies fr1, fr2, . . . , frn so as to generate drive currents id1, id2, . . . , idn, and outputs the drive currents id1, id2, . . . , idn to the light sources 1-1, 1-2, . . . , 1-n, respectively. FIG. 3 is a diagram illustrating an example of the drive current output to the light source in the first embodiment.

In this way, the wavelengths of the measurement light beams Lm1, Lm2, . . . , Lmn generated from the light sources 1-1, 1-2, . . . , 1-n are periodically increased respectively at periods 1/fr1, 1/fr2, . . . , 1/frn within the wavelength ranges determined by the mean current values ic1, ic2, . . . , icn, and the amplitudes irw1, irw2, . . . , irwn of the ramp wave currents.

The controller 9 includes a storage 92. The storage 92 is a part of storage area of the storage device of the computer system. The storage 92 stores various data, various setting values, programs, reference data (described later), and the like.

The storage 92 stores a calibration curve. The calibration curve is pre-obtained data indicating a relationship between a concentration of the target gas and analysis data (described later) (“peak to bottom” value of the analysis data in this embodiment). In this embodiment, there are a plurality of target gases, so the calibration curve is separately obtained for each of the target gases and stored in the storage 92. In addition, the storage 92 stores a reference intensity (described later).

The controller 9 includes a data obtaining unit 93. The data obtaining unit 93 converts a voltage signal or a current signal output from the light receiving device 73 that receives the plurality of measurement light beams Lm1, Lm2, . . . , Lmn (the multiplexed measurement light beam Lm) after passing through the measurement space S into numerical value data that can be recognized by an analyzing unit 94 (described later), by A/D conversion, for example.

The controller 9 includes the analyzing unit 94. The analyzing unit 94 uses data indicating the intensity of the multiplexed measurement light beam Lm obtained by the data obtaining unit 93, as the total intensity Im, so as to analyze the target gases that possibly exist in the measurement space S.

For this purpose, the analyzing unit 94 generates the total intensity Im from the numerical value data obtained by the data obtaining unit 93. Specifically, the analyzing unit 94 generates the total intensity Im that is data in which the second time derivative value of intensity of the multiplexed measurement light beam Lm and the time when the second derivative value is obtained are associated with each other.

Specifically, the analyzing unit 94 first multiplies the numerical value data obtained by the data obtaining unit 93 and the sine wave signal data that is synchronized with the modulation current imod and has a frequency twice the frequency fm of the modulation current imod. Then, a DC component that does not change over time is extracted from the data obtained by the multiplication at a predetermined period. Finally, the total intensity Im is calculated by associating the extracted DC component and the time when the DC component is obtained with each other. This method of obtaining the total intensity Im is referred to as “phase-sensitive detection”.

As the analyzing unit 94 calculates the total intensity Im using the “phase-sensitive detection”, the analyzing unit 94 can analyze the target gases by a wavelength modulation spectrum method (WMS method) using temporal changes of the measured target intensity Id and the reference intensity Is. As a result, even if an influence of the light absorption by the target gas on the measured target intensity Id is small, the target gas can be accurately analyzed.

The analyzing unit 94 analyzes the target gases based on a difference between the measured target intensity Id and the reference intensity Is. The measured target intensity Id is the total intensity Im measured by the light detector 7 when the plurality of measurement light beams Lm1, Lm2, . . . , Lmn (the multiplexed measurement light beam Lm) pass through the measurement space S in which one of the plurality of target gases exists. The reference intensity Is is the total intensity Im measured by the light detector 7 when the plurality of measurement light beams Lm1, Lm2, . . . , Lmn (the multiplexed measurement light beam Lm) pass through the measurement space S in which none of the plurality of target gases exists. Note that a method of analyzing the target gas by the analyzing unit 94 will be described later.

The analyzing unit 94 outputs the analysis result of the target gas (for example, a calculation result of the concentration of the target gas) to the process controller CH1. In this way, the process controller CH1 adjusts the introducing amount of the target gas to be introduced into the chamber CH based on the input analysis result of the target gas, so that an atmosphere in the measurement space S can be adjusted.

The controller 9 includes a temperature controller 95. The temperature controller 95 adjusts the current or the voltage to be output to each of the temperature controllers 11-1, 11-2, . . . , 11-n so that each temperature of the plurality of light sources 1-1, 1-2, . . . , 1-n becomes constant. Specifically, the temperature controller 95 adjusts the current to be output to each of the Peltier devices of the temperature controllers 11-1, 11-2, . . . , 11-n by feedback control so that the temperature measured by the thermistor of the temperature controller 11-1, 11-2, . . . , 11-n becomes a predetermined temperature, for example.

In this embodiment, the process controller CH1 is connected to the controller 9. Therefore the controller 9 includes a gas type reporting unit 96. The gas type reporting unit 96 uses the signal input from the process controller CH1 so as to determine which target gas exists in the measurement space S, and reports the result to the analyzing unit 94.

(1-3) Operation of Gas Analyzing Apparatus

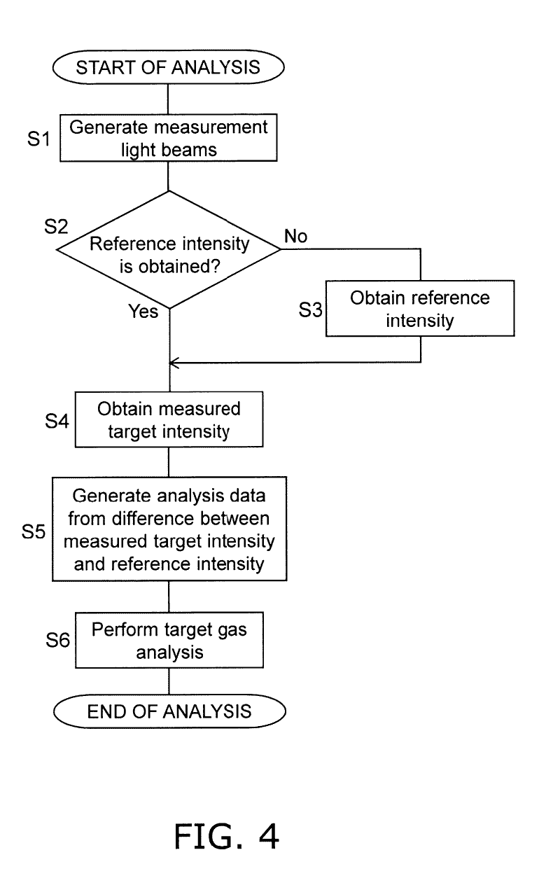

An operation (analyzing process) of the gas analyzing apparatus 100 according to the first embodiment is described below with reference to FIG. 4. FIG. 4 is a flowchart illustrating an operation of the gas analyzing apparatus according to the first embodiment. In the description below, there is described an example of analyzing three types of target gases (gas A, gas B, and gas C) that can exist in the measurement space S. In addition, it is supposed that the number of the light sources is three (light sources 1-1, 1-2, . . . , 1-3).

First, the light source controller 91 outputs the drive currents id1, id2, . . . , id3 to the light sources 1-1, 1-2, . . . , 1-3 so that the measurement light beams Lm1, Lm2, . . . , Lm3 are generated (Step S1).

After that, the analyzing unit 94 determines whether or not the reference intensity Is has been obtained (Step S2). Specifically, the analyzing unit 94 determines whether or not the reference intensity Is is stored in the storage 92.

If it is determined that the reference intensity Is is stored in the storage 92 and that the reference intensity Is has been obtained (“Yes” in Step S2), the analyzing process proceeds to Step S4, and the analyzing unit 94 starts the gas analysis.

On the other hand, if the reference intensity Is is not stored in the storage 92 and therefore it is required to obtain the reference intensity Is (“No” in Step S2), the analyzing unit 94 performs the process to obtain the reference intensity Is (Step S3). Specifically, the analyzing unit 94 obtains the total intensity Im of the multiplexed measurement light beam Lm that has passed through the measurement space S when the gas type reporting unit 96 has not reported introduction of any of the gases A, B, and C (no gas exists in the measurement space S) using the “phase-sensitive detection”, as the reference intensity Is.

Then, the analyzing unit 94 stores the reference intensity Is obtained as described above in the storage 92.

Note that the reference intensity Is is not necessarily obtained during execution of the analyzing process. Since the reference intensity Is is obtained at least before starting the gas analysis, the reference intensity Is may be obtained when performing zero calibration of the gas analyzing apparatus 100, for example.

After obtaining the reference intensity Is, the analyzing unit 94 performs the gas analysis. The analyzing unit 94 first obtains the total intensity Im of the multiplexed measurement light beam Lm as one of the measurement light beams Lm1, Lm2, Lm3 is absorbed when passing through the measurement space S in which one of the gases A to C as the target gases exists, as the measured target intensity Id (Step S4). After that, the analyzing unit 94 stores the obtained measured target intensity Id in the storage 92.

When obtaining the measured target intensity Id, the analyzing unit 94 obtains information about a type of the gas that is introduced into the measurement space S from the gas type reporting unit 96, and associates the obtained measured target intensity Id with the information about the gas type obtained from the gas type reporting unit 96.

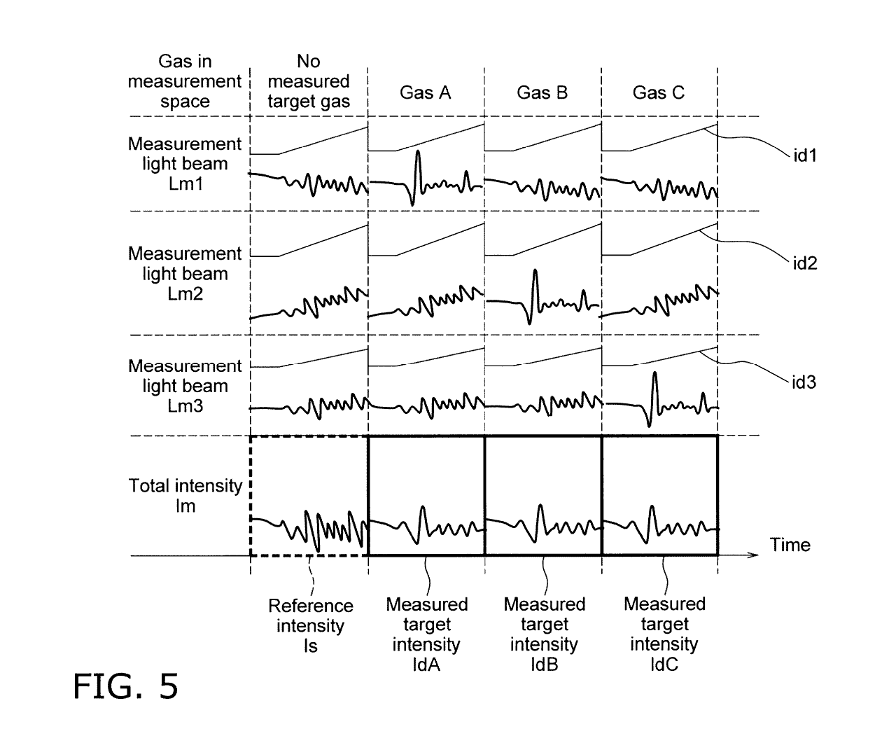

For example, as illustrated in FIG. 5, if the gas A exists in the measurement space S, the measurement light beam Lm1 is absorbed by the gas A, and hence a measured target intensity IdA is obtained. If the gas B exists in the measurement space S, the measurement light beam Lm2 is absorbed by the gas B, and hence a measured target intensity IdB is obtained. If the gas C exists in the measurement space S, the measurement light beam Lm3 is absorbed by the gas C, and hence a measured target intensity IdC is obtained.

FIG. 5 is a diagram illustrating an example of profiles of the measurement light beams and the total intensity in a case where each gas exists.

As illustrated in FIG. 5, the measured target intensity Id measured by the light detector 7 may include intensity components due to influences other than the light absorption by the target gas, for example, intensity components affected by characteristics unique to the gas analyzing apparatus 100. Such characteristics may include characteristics (non-linearity) of the light sources 1-1, 1-2, . . . , 1-n, or characteristics (non-linearity) of the light detector 7. In this way, in the gas analyzing apparatus 100, intensity peaks when the measurement light beams Lm1, Lm2, Lm3 are absorbed by the target gas cannot be recognized accurately.

Accordingly, prior to performing the gas analysis, the analyzing unit 94 eliminates intensity components due to influences other than the light absorption by the target gas from the measured target intensity Id, so as to generate the analysis data that is used for the gas analysis. Specifically, the analyzing unit 94 calculates a difference between the measured target intensity Id obtained in Step S4 and the reference intensity Is stored in the storage 92, as the analysis data (Step S5).

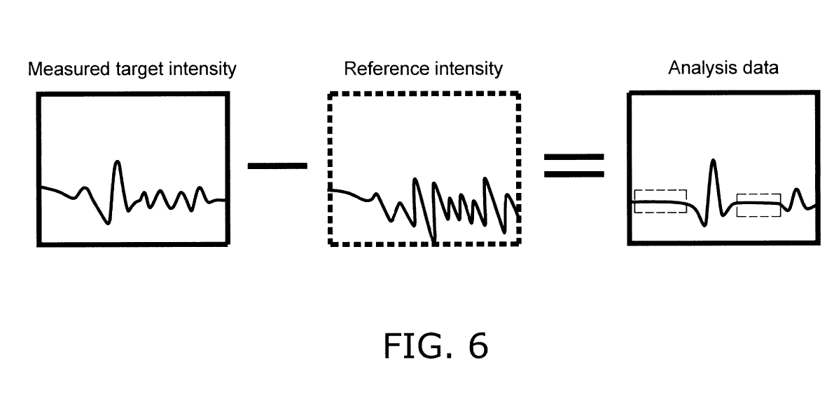

As illustrated in FIG. 6, using the difference between the measured target intensity Id and the reference intensity Is as the analysis data, it is possible to obtain the analysis data in which the reference intensity Is is eliminated from the measured target intensity Id. FIG. 6 is a diagram schematically illustrating a method of calculating the analysis data.

After generating the analysis data, the analyzing unit 94 performs analysis of the target gases (gases A to C) by using the analysis data (Step S6). Specifically, the analyzing unit 94 calculates concentrations of gases A to C in the measurement space S as follows.

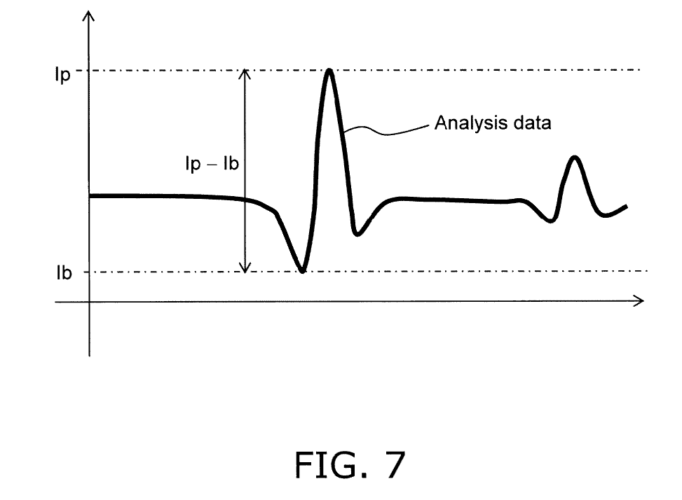

First, the analyzing unit 94 calculates the “peak to bottom” value of the analysis data. For example, as illustrated in FIG. 7, a difference Ip−Ib between the maxima Ip that is maximum in the analysis data and the minima Ib that is minimum in the same is calculated as the “peak to bottom” value. FIG. 7 is a diagram illustrating an example of a method of calculating the “peak to bottom” value from the analysis data.

Next, the analyzing unit 94 reads the calibration curve of the gas to be analyzed from the storage 92 and calculates the concentration of the gas having the “peak to bottom” value that is the difference Ip−Ib in the calibration curve. Specifically, for example, if the calibration curve is expressed by Y=AX+B, where Y is the “peak to bottom” value, X is the concentration of the gas, and A and B are constant values, the concentration of the gas is calculated to be (Ip−Ib−B)/A by substituting Ip−Ib for Y in the equation and then solving the equation for X.

Note that the gas/gasses calibration curve which is read from the storage 92 can be determined from information about the gas type associated with the measured target intensity Id from which the analysis data is calculated.

As described above, in the gas analyzing apparatus 100, both the reference intensity Is and the measured target intensity Id are based on the intensities (total intensity Im) of the plurality of measurement light beams Lm1, Lm2, Lm3 generated simultaneously from the plurality of light sources 1-1, 1-2, 1-3. In this way, the gas analyzing apparatus 100 can accurately analyze a plurality of target gases without providing light sources respectively for the target gases to measure the reference intensity Is.

In addition, in the gas analyzing apparatus 100, the controller 9 is connected to the process controller CH1, and the gas type reporting unit 96 is disposed. In this way, even if a type of the target gas that exists in the measurement space S cannot be determined from the analysis data calculated from the measured target intensity Id (for example, even if the measured target intensity (analysis data) has no dependency on the gas type), it is possible to detect which target gas exists in the measurement space S.

### (2) Second Embodiment

In the first embodiment, the inlet 5 introduces the multiplexed measurement light beam Lm into only one measurement space S. However, if the inlet 5 can at least introduce the multiplexed measurement light beam Lm into the measurement space S, the number of outlets for the multiplexed measurement light beam Lm is not limited.

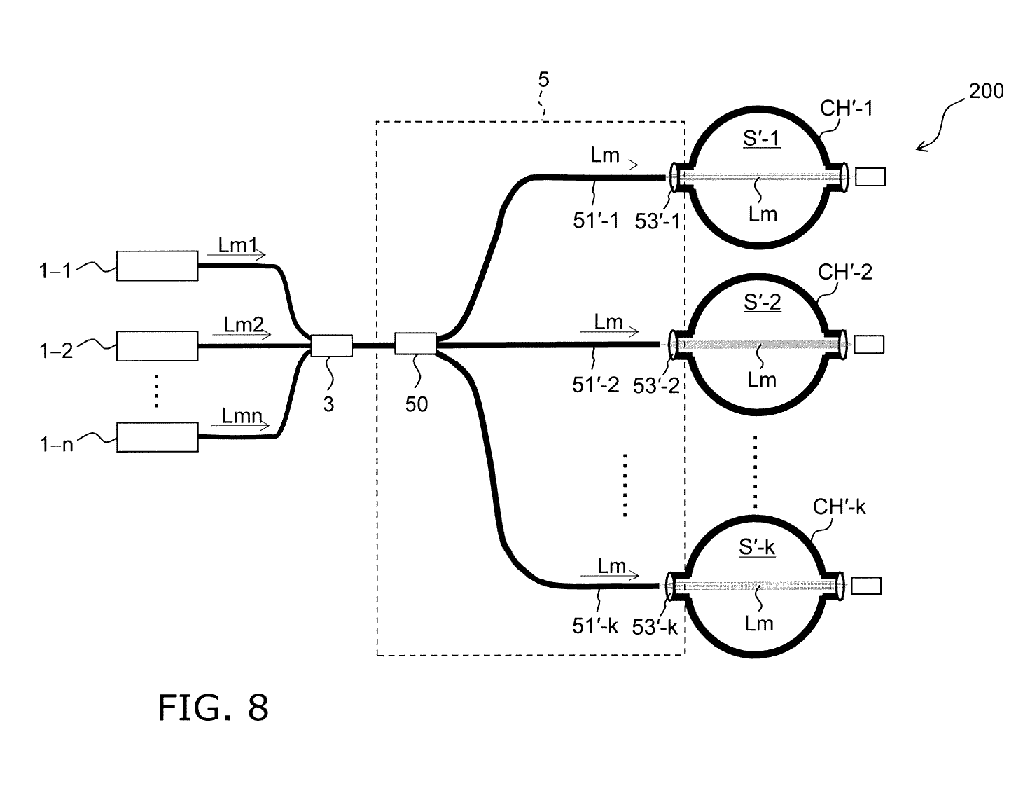

In a gas analyzing apparatus 200 according to the second embodiment, the inlet 5 includes a light splitter 50 that is a fiber coupler that splits the multiplexed measurement light beam Lm so as to output the same from a plurality of light outlets 51′-1, 51′-2, . . . , 51′-k as illustrated in FIG. 8. FIG. 8 is a diagram illustrating a structure of the gas analyzing apparatus according to the second embodiment.

Specifically, as illustrated in FIG. 8, the plurality of light outlets 51′-1, 51′-2, . . . , 51′-k as optical fibers are extended from an end of the light splitter 50. The plurality of light outlets 51′-1, 51′-2, . . . , 51′-k are fused inside the light splitter 50 and are connected to the outlet of the multiplexed measurement light beam Lm of the optical multiplexer 3. In this way, the multiplexed measurement light beam Lm output from the optical multiplexer 3 propagates in the plurality of light outlets 51′-1, 51′-2, . . . , 51′-k and are output from ends opposite to the fused ends of the plurality of light outlets 51′-1, 51′-2, . . . , 51′-k, respectively.

As described above, because the multiplexed measurement light beam Lm is output from the plurality of light outlets 51′-1, 51′-2, . . . , 51′-k, as illustrated in FIG. 8, for example, each of the plurality of multiplexed measurement light beams Lm output from the plurality of light outlets 51′-1, 51′-2, . . . , 51′-k can be simultaneously introduced into each of the plurality of measurement spaces S′-1, S′-2, . . . , S′-k, respectively, via the collimator 53′-1, 53′-2, . . . , 53′-k disposed in the openings of the plurality of chambers CH′-1, CH′-2, . . . , CH′-k.

By simultaneously introducing the multiplexed measurement light beam Lm into the plurality of measurement spaces S′-1, S′-2, . . . , S′-k, it is not necessary to dispose light sources respectively for the measurement spaces to analyze the target gases in the plurality of measurement spaces S′-1, S′-2, . . . , S′-k.

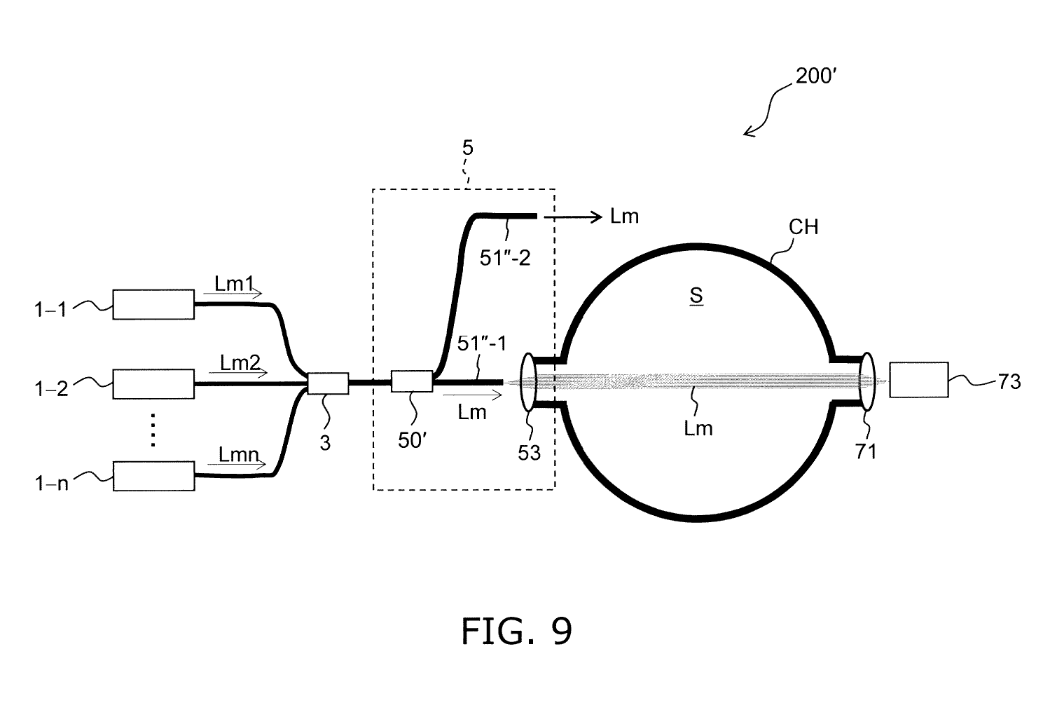

In a gas analyzing apparatus 200′ as a variation of the second embodiment, the inlet 5 includes a light splitter 50′ that outputs the multiplexed measurement light beam Lm from two light outlets 51″-1, 51″-2 as illustrated in FIG. 9. FIG. 9 is a diagram illustrating a structure of a gas analyzing apparatus as a variation of the second embodiment.

In this case, an outlet of one light outlet 51″-1 is disposed in the vicinity of the collimator 53, and the multiplexed measurement light beam Lm output from the light outlet 51″-1 is introduced into the measurement space S. On the other hand, an outlet of another light outlet 51″-2 is disposed in the external space. In other words, the multiplexed measurement light beam Lm that has propagated in the light outlet 51″-2 is output to the external space.

Because the multiplexed measurement light beam Lm can be externally output, when the gas analyzing apparatus 200′ malfunctions, the cause of the malfunction can be detected easily. In addition, by measuring intensity of the multiplexed measurement light beam Lm output externally, the intensity of the multiplexed measurement light beam Lm output externally can be used as the reference intensity Is described above in the first embodiment.

### (3) Third Embodiment

In the first embodiment and the second embodiment, the direction in which the drive currents id1, id2, . . . , idn input to the light sources 1-1, 1-2, . . . , 1-n increase or decrease over time, and the timing at which the drive current rapidly changes from the maximum value to the minimum value (periodically increasing/decreasing timing of the drive current) are the same for all the light sources. In other words, the measurement light beams Lm1, Lm2, . . . , Lmn have the same pattern of periodical change. However, without limiting to this, in a gas analyzing apparatus 300 according to the third embodiment, a periodic increase/decrease pattern of some measurement light beams among the plurality of measurement light beams Lm1, Lm2, . . . , Lmn may be different from a periodic increase/decrease pattern of other measurement light beams.

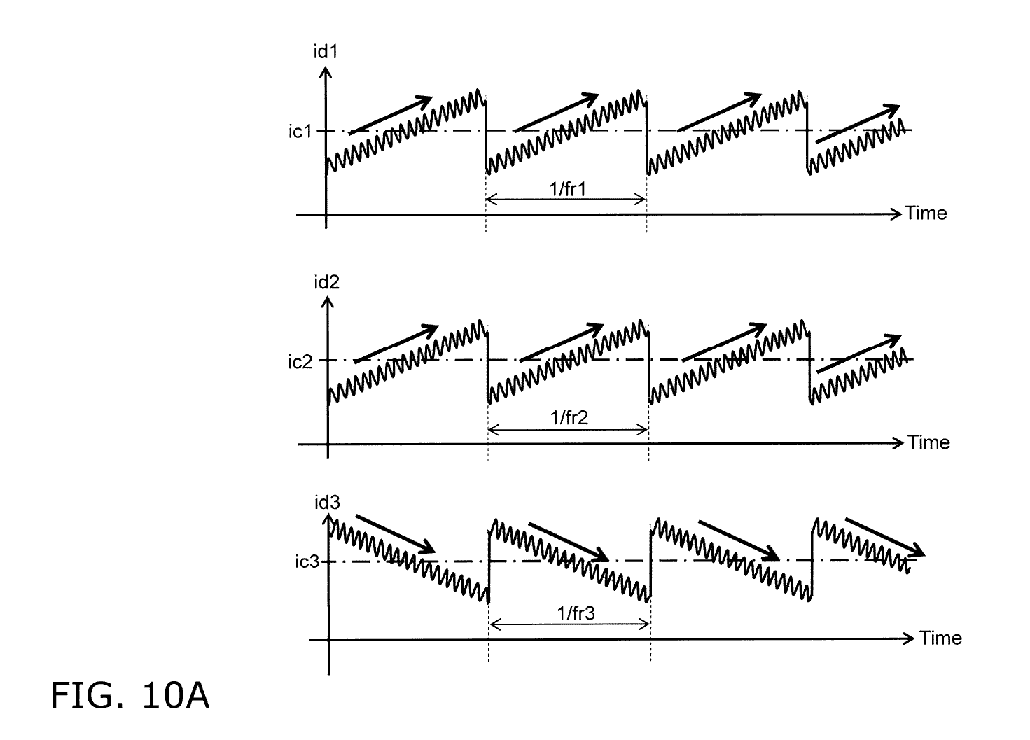

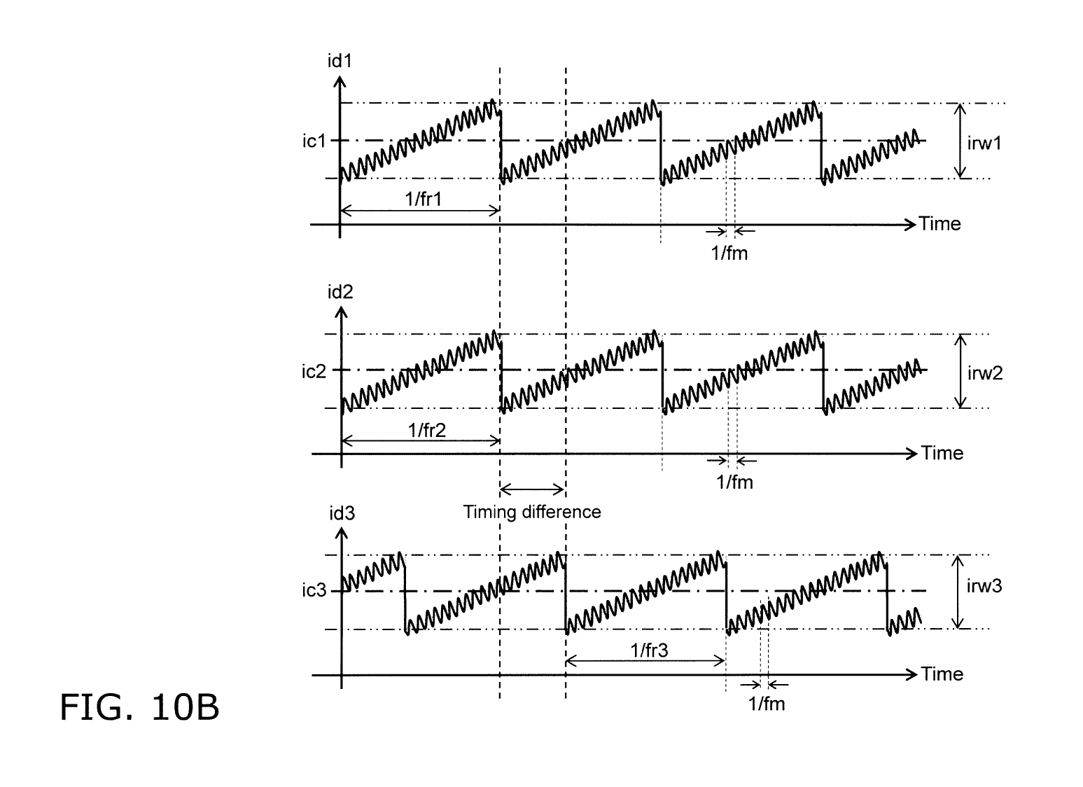

For example, as illustrated in FIG. 10A, it is possible to periodically decrease the drive current id3 input to the light source 1-3 at period 1/fr3, and to periodically increase the drive current id1, id2 of the other light sources 1-1, 1-2 at periods 1/fr1, 1/fr2. In this way, the intensities of the measurement light beams Lm1, Lm2 are periodically increased at periods 1/fr1, 1/fr2, respectively, while the intensity of the measurement light beam Lm3 is periodically decreased at period 1/fr3. FIG. 10A is a diagram illustrating an example of the drive current output to the light source of the third embodiment.

In this way, by periodically increasing the intensity of any measurement light beam while periodically decreasing the intensity of another measurement light beam, it is possible to reduce the total light amount of the plurality of measurement light beams, so that a dynamic range in analyzing the target gases can be increased.

The increase/decrease patterns of the plurality of measurement light beams are not limited to those illustrated in FIG. 10A, if the total light amount can at least be decreased. For example, it is possible to periodically increase the intensity of the measurement light beam Lm1 of the light source 1-1, and to periodically decrease the intensities of the measurement light beams Lm2, Lm3 of the other light sources 1-2, 1-3. Alternatively, it is possible to periodically increase the intensity of the measurement light beam Lm2 of the light source 1-2, and to periodically decrease the intensities of the measurement light beams Lm1, Lm3 of the other light sources 1-1, 1-3. Further, it is possible to periodically increase the intensity of the measurement light beam Lm3 of the light source 1-3 and to periodically decrease the intensities of the measurement light beams Lm1, Lm2 of the other light sources 1-1, 1-2.

In addition, as illustrated in FIG. 10B, for example, it is possible to differentiate between the timing of the drive current id3 input to the light source 1-3 and the timing of the drive currents id1, id2 input to the other light sources 1-1, 1-2. FIG. 10B is a diagram illustrating another example of the drive currents output to the light sources of the third embodiment. Alternatively, it is possible to differentiate among the timings of the drive currents id1, id2, id3 input to the light sources 1-1, 1-2, 1-3.

Some or all of the timings of the drive current id1, id2, . . . , idn input to the light sources 1-1, 1-2, . . . , 1-n may be differentiated from each other so that periodic increase/decrease phases of some measurement light beams are differentiated from periodic increase/decrease timings of the other measurement light beams, and hence a total light amount of the plurality of measurement light beams can be reduced to increase the dynamic range in analyzing the target gases.

Further, also by differentiating increase/decrease directions of some of the drive currents input to the light sources 1-1, 1-2, . . . , 1-n from increase/decrease directions of other drive currents, and by differentiating periodic increase/decrease timings of some of the drive currents from periodic increase/decrease timings of other drive currents, it is possible to reduce the total light amount of the plurality of measurement light beams.

In addition, by differentiating among the temporal increase/decrease directions of the drive current id1, id2, . . . , idn input to the light sources 1-1, 1-2, . . . , 1-n, and/or by differentiating among the periodic increase/decrease timings of the drive current id1, id2, . . . , idn, even if the target gas type cannot be determined from the profile of the analysis data when the patterns of periodical changes of the measurement light beams Lm1, Lm2, . . . , Lmn are the same, it is possible to determine the gas type that exists in the measurement space S from the profile of the analysis data.

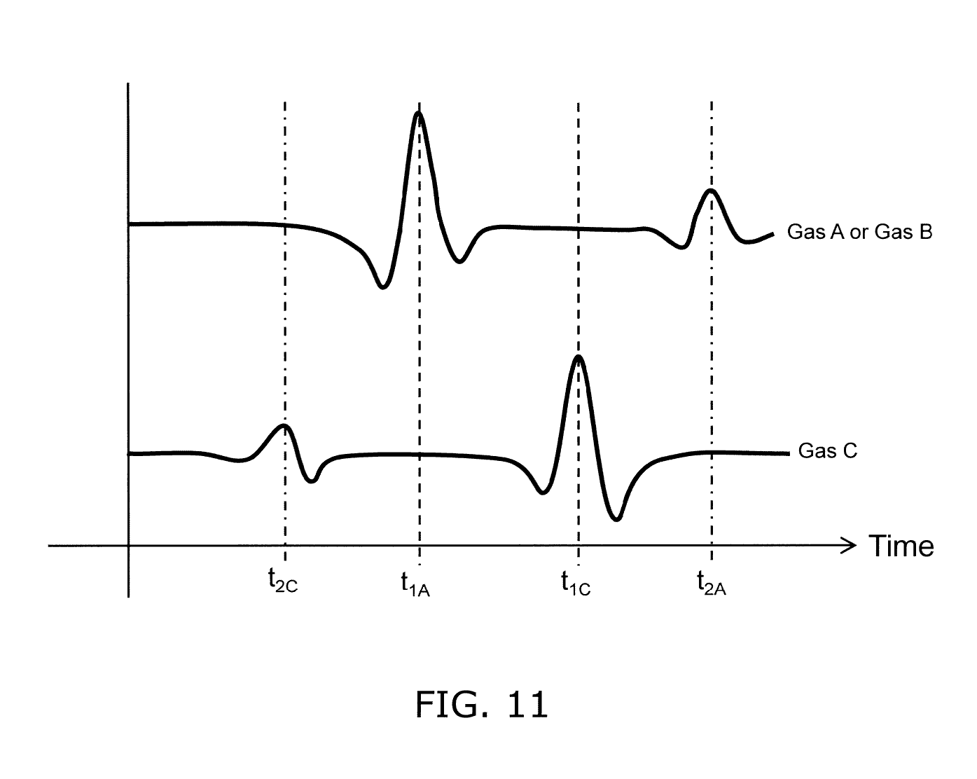

For example, as illustrated in FIG. 11, peak occurrence timing (t_{1A}, t_{2A}) of the analysis data when the gas A or B exists in the measurement space S can be differentiated from peak occurrence timing (t_{1C}, t_{2C}) of the analysis data when the gas C exists in the measurement space S. FIG. 11 is a diagram illustrating an example of gas type dependency of the profile of the analysis data, which is obtained by differentiating among the patterns of periodical changes of the plurality of measurement light beams.

### (4) Fourth Embodiment

### (4-1) Outline of Gas Analyzing Apparatus According to Fourth Embodiment

In the conventional gas analyzing apparatus that analyzes a gas in a measurement space using light absorption of a measurement light beam, the target gas in the measurement space is analyzed based on the peak value in the analysis data generated due to the light absorption by the target gas. Conventionally, the analysis data that is used for gas analysis is generated by subtracting the intensity of the measurement light beam that has passed through the measurement space in which the target gas does not exist (the reference intensity) from the intensity of the measurement light beam that has passed through the measurement space in which the target gas to be analyzed exists.

In the analysis data calculated in this way, if the concentration of the target gas in the measurement space is low, the peak value included in the analysis data tends to be small, or the peak due to the light absorption cannot be clearly recognized in the analysis data. As a result, the conventional gas analyzing apparatus cannot accurately analyze the target gas having a low concentration by using the analysis data.

A gas analyzing apparatus 400 according to the fourth embodiment can accurately analyze the target gas having a low concentration in particular, by using the analysis data, which is generated by subtracting the intensity of the measurement light beam that has passed through the measurement space in which a concentration of the target gas exists is low, from the intensity of the measurement light beam that has passed through the measurement space in which the concentration of the target gas is high.

### (4-2) Overall Structure of Gas Analyzing Apparatus According to Fourth Embodiment

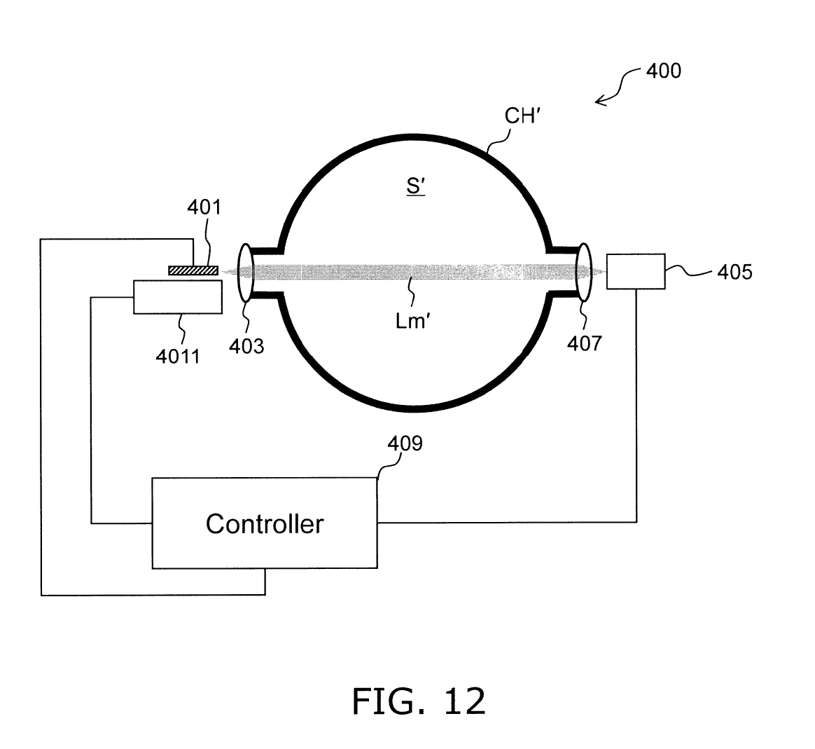

The gas analyzing apparatus 400 according to the fourth embodiment is described below in detail. First, an overall structure of the gas analyzing apparatus 400 according to the fourth embodiment is described with reference to FIG. 12. FIG. 12 is a diagram illustrating an overall structure of the gas analyzing apparatus according to the fourth embodiment.

The gas analyzing apparatus 400 is an apparatus that analyzes a target gas that possibly exists in a measurement space S′ that is an inner space of a chamber CH′.

The gas analyzing apparatus 400 includes a light source 401. The light source 401 is a laser diode (LD), for example, which outputs a measurement light beam Lm′ having a unique wavelength range that is absorbed by the target gas. Note that the number of the light sources provided to the gas analyzing apparatus 400 is not limited to one. As described above in the first embodiment, the gas analyzing apparatus 400 may be equipped with a plurality of light sources.

The measurement light beam Lm′ generated from the light source 401 enters a collimator 403 (an example of the inlet) attached to an opening of the chamber CH′. The measurement light beam Lm′ entering the collimator 403 is collimated and the collimated measurement light beam Lm′ is introduced into the measurement space S′.

A temperature controlling apparatus 4011 is attached to the light source 401. The temperature controlling apparatus 4011 can include a Peltier device and a temperature sensor (e.g. a thermistor), for example.

The temperature controlling apparatus 4011 adjusts temperature of the light source 401 to a constant value. In this way, the light source 401 can control wavelength of the measurement light beam Lm′ based on an input current level without being affected by the surrounding or ambient temperature.

The gas analyzing apparatus 100 includes a light detector 405. The light detector 405 measures an intensity Im′ of the measurement light beam Lm′ that has been introduced into the measurement space S′ from the collimator 403 and passed through the measurement space S′.

The light detector 405 is a photodiode or a photomultiplier tube, which is disposed in the vicinity of a light collector 407 such as a lens attached to an opening disposed to face the opening to which the collimator 403 is attached.

In this way, the light detector 405 can receive the measurement light beam Lm′ that has passed through the measurement space S′ and been condensed by the light collector 407. The light detector 405 generates an electric signal based on the intensity of the measurement light beam Lm′ received by a light receiving surface, and outputs the electric signal to a controller 409 (described later).

Note that the measurement light beam Lm′ introduced into the measurement space S′ may be received by the light detector 405 after being reflected (a plurality of times) in the measurement space S′ by a (plurality of) reflector(s) (not shown) disposed in the measurement space S′ and/or an inner wall of the chamber CH′.

The gas analyzing apparatus 400 includes the controller 409. The controller 409 is a computer system including a CPU, a storage device (a device that stores data such as a RAM and a ROM or the like), and various interfaces (such as an A/D converter, a D/A converter, and a communication circuitry). The controller 409 controls the elements of the gas analyzing apparatus 400. The controller 409 performs various information processes to analyze the target gas in the measurement space S′.

A structure of the controller 409 and information processes performed by the controller 409 will be described later.

With the structure described above, the gas analyzing apparatus 400 can analyze the target gas in the measurement space S′ based on intensity of the measurement light beam Lm′ that has passed through the measurement space S′ inside the chamber CH′ and been received by the light detector 405.

(4-3) Structure of Controller

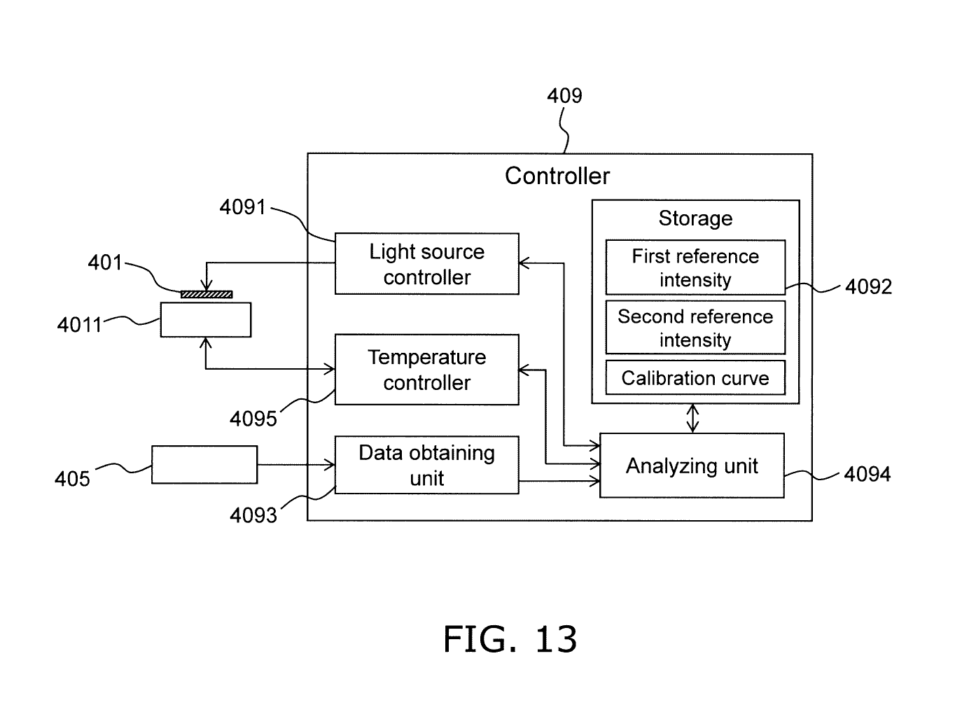

Next, a structure of the controller 409 is described with reference to FIG. 13. FIG. 13 is a diagram illustrating a structure of a controller according to the fourth embodiment. Note that a part of the functions of individual elements of the controller 409 described below may be realized by a program that is stored in the storage device and is executed by the computer system constituting the controller 409. A part of functions of individual elements of the controller 409 may be realized by a custom IC.

The controller 409 includes a light source controller 4091. As illustrated in FIG. 3, the light source controller 4091 outputs to the light source 401 the drive current id1 that is, for example, a current obtained by summing the mean current ic1 that determines a center wavelength of the wavelength range of the measurement light beam output from the light source 401, the ramp wave current ir1 that has the amplitude irw1 and the frequency fr1, and the modulation current imod that is a sine wave having a frequency fm higher than the frequency fr1.

In this way, the wavelength of the measurement light beam Lm′ generated from the light source 401 is periodically increased at period 1/fr1 within the wavelength range determined by a value of the mean current ic1 and the amplitude irw1 of the ramp wave current.

The controller 409 includes a storage 4092. The storage 4092 is a part of the storage area of the storage device of the computer system. The storage 4092 stores various data, various setting values, programs, a first reference intensity (described later), a second reference intensity, and the like.

The storage 4092 stores a calibration curve. The calibration curve is pre-obtained data indicating a relationship between a concentration of the target gas and the analysis data (“peak to bottom” value of the analysis data in this embodiment).

The controller 409 includes a data obtaining unit 4093. The data obtaining unit 4093 converts a voltage signal or a current signal output from the light detector 405 after receiving the measurement light beam Lm′ that has passed through the measurement space S′ into numerical value data that can be recognized by an analyzing unit 4094 (described later) by an A/D conversion, for example.

The controller 409 includes the analyzing unit 4094. The analyzing unit 4094 uses data indicating intensity of the measurement light beam Lm′ obtained by the data obtaining unit 4093, as the intensity Im′, so as to analyze the target gas that exists in the measurement space S′.

For this purpose, the analyzing unit 4094 generates the intensity Im′ from the numerical value data obtained by the data obtaining unit 4093. Specifically, the analyzing unit 4094 generates the intensity Im′ that is the data in which the second time derivative value of the intensity of the measurement light beam Lm′ and the time when the second derivative value is obtained are associated with each other.

Specifically, the analyzing unit 4094 first multiplies the numerical value data obtained by the data obtaining unit 4093 and the sine wave signal data that is synchronized with the modulation current imod and has a frequency twice the frequency fm of the modulation current imod. Then, a DC component that does not change over time is extracted from the data obtained by the multiplication at a predetermined period. Finally, the extracted DC component and the time when the DC component is obtained are associated with each other and calculated as the intensity Im′ (phase-sensitive detection).

As the analyzing unit 4094 calculates the intensity Im′ by using the “phase-sensitive detection”, the analyzing unit 4094 can analyze the target gas by the wavelength modulation spectrum method (WMS method) using temporal changes of the measured target intensity Id′ and the reference intensity (the first reference intensity Is1′, the second reference intensity Is2′). As a result, even if an influence of the light absorption by the target gas on the measured target intensity Id′ is small, the target gas can be accurately analyzed.

The analyzing unit 4094 analyzes the target gas in the measurement space S′ by using the analysis data. In this embodiment, the analyzing unit 4094 differentiates the method of generating the analysis data between before and after the concentration of the target gas in the measurement space S′ becomes the first concentration. The method of generating the analysis data and the method of analyzing the target gas in the analyzing unit 4094 will be described later.

The controller 409 includes a temperature controller 4095. The temperature controller 4095 adjusts current or voltage to be output to the temperature controlling apparatus 4011 so that temperature of the light source 401 becomes constant. Specifically, for example, the temperature controller 4095 adjusts the current to be output to the Peltier device of the temperature controlling apparatus 4011 by feedback control so that the temperature measured by the thermistor of the temperature controlling apparatus 4011 becomes a predetermined temperature.

(4-4) Operation of Gas Analyzing Apparatus According to Fourth Embodiment

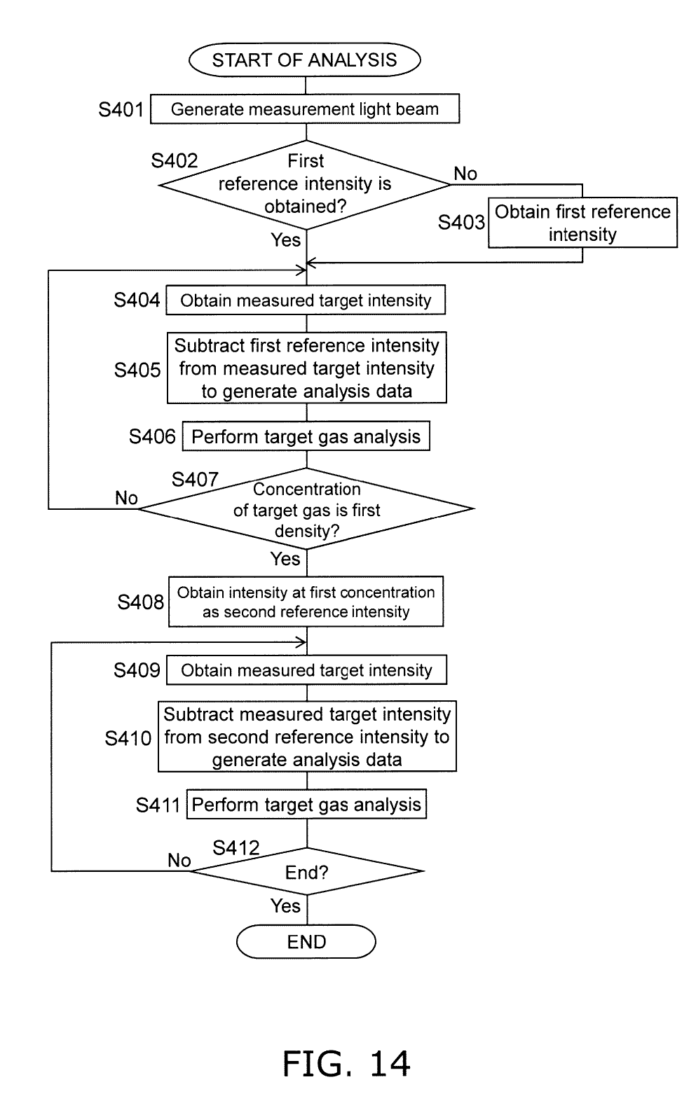

An operation (analyzing process) of the gas analyzing apparatus 400 according to the fourth embodiment is described below with reference to FIG. 14. FIG. 14 is a flowchart illustrating an operation of the gas analyzing apparatus according to the fourth embodiment.

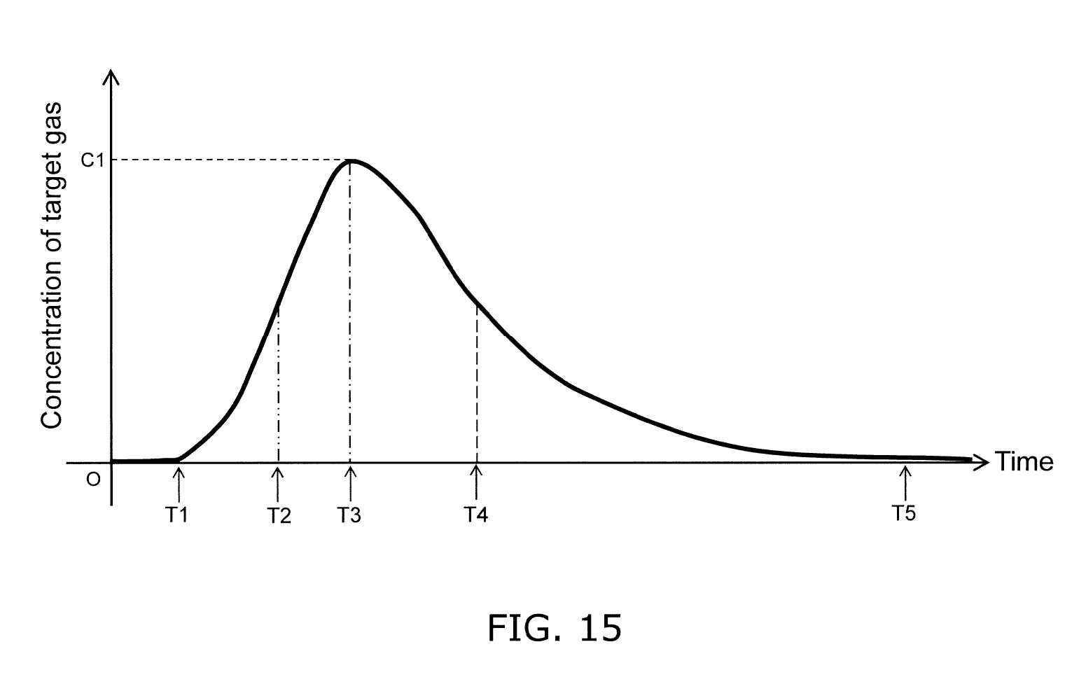

In the description below, the process of analyzing the target gas in which the concentration of the target gas in the measurement space S′ changes as time proceeds is illustrated. FIG. 15 is a diagram illustrating an example of a temporal variation of the concentration of the target gas.

In the example illustrated in FIG. 15, for example, as the target gas is introduced into the chamber CH′ at time point T1 and the concentration of the target gas in the measurement space S′ is increased after the time point T1. After that, for example, when the introduction of the target gas into the chamber CH′ is stopped, the concentration of the target gas in the measurement space S′ is gradually decreased after reaching the first concentration C1 at time point T3. In this example, the first concentration C1 at time point T3 is the maximum concentration of the target gas.

When the gas analyzing apparatus 400 starts the target gas analyzing process, the light source controller 4091 first outputs the drive current id1 to the light source 401 so that the measurement light beam Lm′ is generated (Step S401).

Then, the analyzing unit 4094 determines whether or not the reference intensity to be referenced when generating the analysis data has been obtained (Step S402). In Step S402, just after the gas analyzing process is started, the analyzing unit 4094 determines that the intensity Im′ of the measurement light beam Lm′, that has passed through the measurement space S′ in which no target gas exists and been received by the light detector 405, is the first reference intensity Is1′ to be used to generate the analysis data.

For example, if the first reference intensity Is1′ is already stored in the storage 4092 and it is determined that the first reference intensity Is1′ has been obtained (“Yes” in Step S402), the analyzing process proceeds to Step S404, and the gas analysis is started.

On the other hand, if the first reference intensity Is1′ is not stored in the storage 4092 and it is determined that the first reference intensity Is1′ is required to be obtained (“No” in Step S402), the analyzing unit 4094 performs the process to obtain the first reference intensity Is1′ (Step S403).

Specifically, before the time point T1 shown in FIG. 15 is passed (in other words, before the target gas is introduced into the chamber CH′), the analyzing unit 4094 obtains the first reference intensity Is1′, which is the intensity Im′ of the measurement light beam Lm′ that has passed through the measurement space S′ by the “phase-sensitive detection” described above. After that, the analyzing unit 4094 stores the first reference intensity Is1′ obtained as described above in the storage 4092.

Note that the first reference intensity Is1′ described above may be obtained, for example, when the zero calibration of the gas analyzing apparatus 400 is performed before the target gas analyzing process is performed, and may be stored in the storage 4092.

After obtaining the first reference intensity Is1′, the analyzing unit 4094 performs the gas analysis. The analyzing unit 4094 first obtains the measured target intensity Id′, which is the intensity Im′ of the measurement light beam Lm′ that is absorbed when passing thorough the measurement space S′ in which the target gas exists (Step S404). Then, the analyzing unit 4094 stores the obtained measured target intensity Id′ in the storage 4092.

After obtaining the measured target intensity Id′, the analyzing unit 4094 calculates the analysis data. The analysis data is the difference between the measured target intensity Id′ obtained in Step S404 as described above and the first reference intensity Is1′ stored in the storage 92 (Step S405).

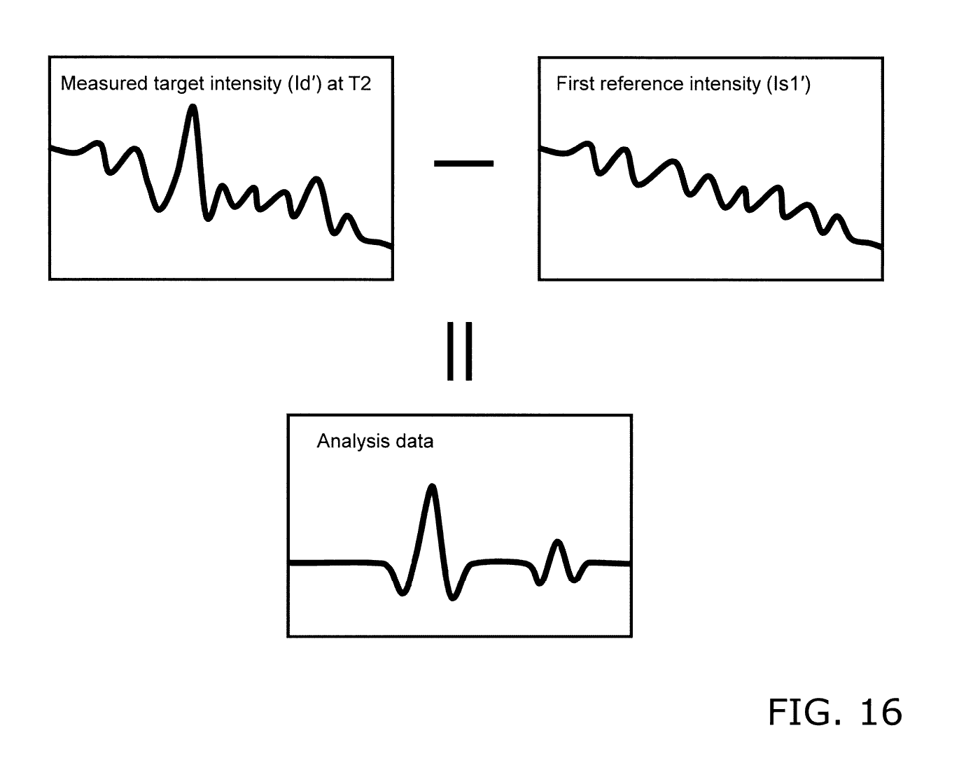

For example, if the measured target intensity Id′ (FIG. 16) is obtained at the time point T2 shown in FIG. 15, the first reference intensity Is1′ (FIG. 16) is subtracted from the measured target intensity Id′, so as to obtain the analysis data without an intensity component affected by characteristics unique to the gas analyzing apparatus 400 as illustrated in FIG. 16. FIG. 16 is a diagram schematically illustrating a method of generating the analysis data.

After generating the analysis data, the analyzing unit 4094 performs the target gas analysis using the generated analysis data (Step S406). In Step S406, the concentration of the target gas existing in the measurement space S′ is calculated using the “peak to bottom” value of the analysis data.

Specifically, for example, if the calibration curve of the target gas is expressed by Y=VX+W, where Y is the “peak to bottom” value, X is the concentration, and V and W are constant values, the analyzing unit 4094 can calculate the concentration of the target gas by substituting the “peak to bottom” value of the analysis data for Y in the equation expressing the calibration curve and the solving the equation for X.

As described above, if the concentration of the target gas in the measurement space S′ is relatively large so that the peak due to the light absorption of the measurement light beam Lm′ by the target gas can also be recognized in the profile of the measured target intensity Id′, the peak due to the light absorption of the measurement light beam Lm′ by the target gas can also be recognized in the analysis data obtained by subtracting the first reference intensity Is1′ from the measured target intensity Id′. In this case, by using the “peak to bottom” value in the analysis data and the calibration curve, the concentration of the target gas can be accurately calculated.

On the other hand, the intensity Im′ (measured target intensity Id′) of the measurement light beam Lm′ that has passed through the measurement space S′ in which the target gas exists at a low concentration like the concentration at the time point T5 shown in FIG. 15 has substantially the same intensity as the first reference intensity Is1′ described above. It is because if the concentration of the target gas existing in the measurement space S′ is low, the light absorption of the measurement light beam Lm′ by the target gas tends to be small.

The analysis data generated by subtracting the first reference intensity Is1′ from the above measured target intensity Id′ has substantially no peak due to the light absorption by the target gas. In other words, when using the analysis data generated by subtracting the first reference intensity Is1′ from the measured target intensity Id′, the target gas having a low concentration cannot be analyzed accurately.

Therefore, in this embodiment, in order to accurately analyze the target gas having a low concentration, the intensity Im′ of the measurement light beam Lm′ that has passed through the measurement space S′ in which the concentration of the target gas is high enough to sufficiently absorb the measurement light beam Lm′ is set as the reference intensity (referred to as the second reference intensity Is2′), and the measured target intensity Id′ is subtracted from the second reference intensity Is2′ so as to obtain the analysis data that is used to analyze the target gas having a low concentration.

In this embodiment, the intensity Im′ obtained at the time point T3 when the concentration of the target gas is maximum (the first concentration C1 (FIG. 15)) is set as the second reference intensity Is2′.

Specifically, during execution of the analyzing process in Steps S404 to S406 as described above, the analyzing unit 4094 determines whether or not the concentration of the target gas calculated in Step S406 has become the first concentration C1 (Step S407).

If it is determined that the concentration of the target gas calculated in Step S406 has not become the first concentration C1 (“No” in Step S407), the analyzing process returns to Step S404, and the target gas analysis in which the first reference intensity Is1′ is set as the reference intensity is continued.

On the other hand, if it is determined that the concentration of the target gas has become the first concentration C1 (“Yes” in Step S407), the analyzing unit 4094 stores the intensity Im′ of the measurement light beam Lm′ obtained when the calculated concentration has become the first concentration C1 (at the time point T3 in the example illustrated in FIG. 15) in the storage 4092 as the second reference intensity Is2′ (Step S408). The second reference intensity Is2′ obtained as described above includes a clear peak due to the light absorption by the target gas.

After obtaining the second reference intensity Is2′ (after the time point T3 in the example illustrated in FIG. 15), the analyzing unit 4094 obtains the measured target intensity Id′ similarly to Step S404 described above (Step S409). The analyzing unit 4094 then subtracts the measured target intensity Id′ from the second reference intensity Is2′ stored in the storage 4092 so as to generate the analysis data (Step S410).

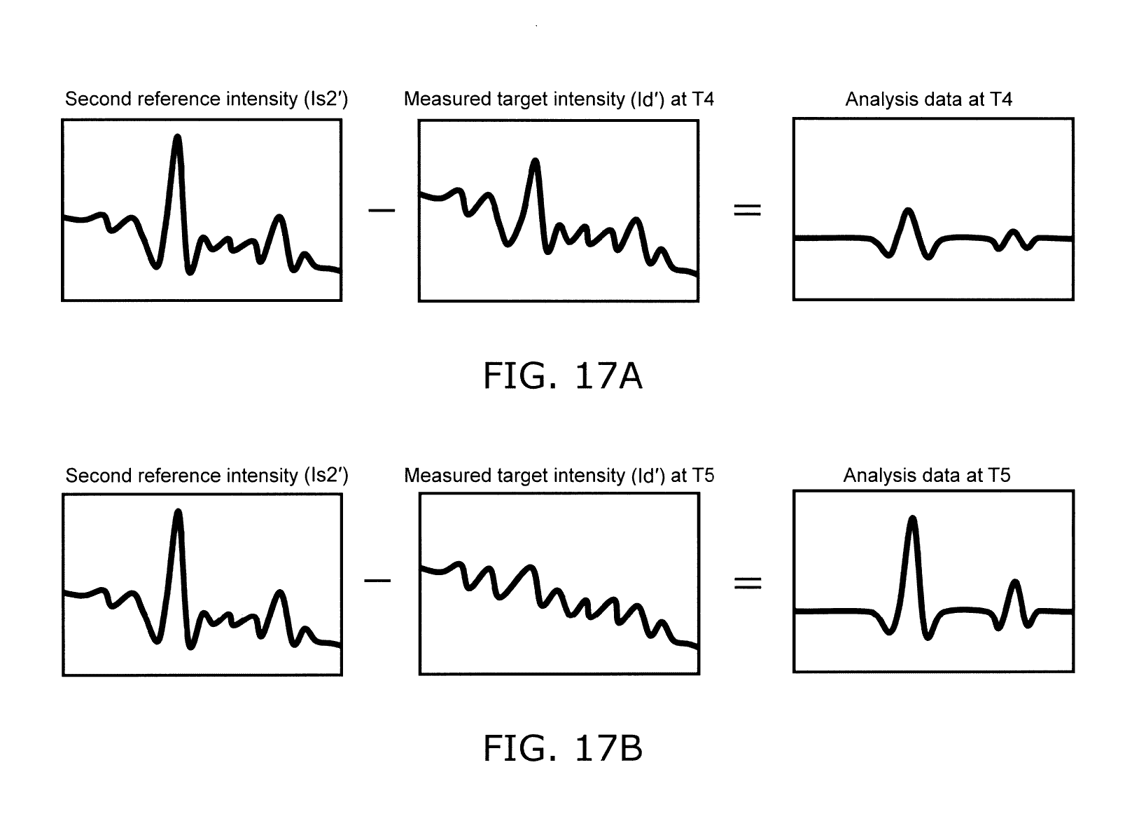

After the time point T3 of FIG. 15, for example, at the time point T4 when the concentration of the target gas becomes the same level as at the time point T2, the analysis data as illustrated in FIG. 17A is generated by subtracting the measured target intensity Id′ (FIG. 17A) from the second reference intensity Is2′. In other words, the analysis data including a peak due to the light absorption by the target gas is obtained.

FIG. 17A is a diagram illustrating an example of the analysis data obtained by subtracting the measured target intensity from the second reference intensity.

When the concentration of the target gas is so low (at the time point T5 in FIG. 15) that the measured target intensity Id′ (FIG. 17B) with a small peak due to the light absorption is obtained, the analysis data as illustrated in FIG. 17B is generated by subtracting the measured target intensity Id′ from the second reference intensity Is2′. In other words, the analysis data including a peak due to the light absorption by the target gas is generated. FIG. 17B is a diagram illustrating another example of the analysis data obtained by subtracting the measured target intensity from the second reference intensity.

After generating the analysis data as described above, the analyzing unit 4094 performs the target gas analysis (calculation of the concentration) using the “peak to bottom” value in the analysis data and the calibration curve described above (Step S411).

Note that, in Step S411, by using the analysis data and the calibration curve, the concentration of the target gas is calculated as a difference between the first concentration C1 and the concentration of the target gas when obtaining the measured target intensity Id′ that is used to generate the analysis data. It is because the analysis data used in Step S411 is generated with reference to the second reference intensity Is2′, which is the intensity Im′ of the measurement light beam Lm′ when the concentration of the target gas is the first concentration C1.

This is also understood, for example, from the fact that the peak of the analysis data illustrated in FIG. 17B (generated at the time point T5 in FIG. 15) is larger than the peak of the analysis data illustrated in FIG. 17A (generated at the time point T4 in FIG. 15).

After the target gas analysis, the controller 409 determines whether or not to finish the analyzing process (Step S412). For example, it determines to finish the analyzing process when the controller 409 receives an analysis end signal or when an analysis end instruction is input by an input device of the controller 409.

When it is determined that the analyzing process is to be stopped (“Yes” in Step S412), the analyzing process is finished. On the other hand, when the analyzing process is not stopped (“No” in Step S412), the analyzing process returns to Step S409, and the target gas analysis is continued.

In the fourth embodiment described above, when analyzing a target gas having a concentration lower than the first concentration C1, the intensity Im′ of the measurement light beam Lm′ having a clear peak due to the light absorption obtained when the concentration is the first concentration C1 is set as the reference intensity (the second reference intensity Is2′) so as to generate the analysis data. In this way, even if sufficient light absorption of the measurement light beam Lm′ cannot be obtained by the target gas having a concentration lower than the first concentration C1, it is possible to generate the analysis data having a clear peak due to the light absorption. In addition, using the analysis data and using fitting calculation or the like, noise reduction calculation can be performed.

As a result, the target gas analysis in the measurement space S′ in which a concentration of a target gas is low can be accurately performed.

(5) Other Embodiments

Although the embodiments of the present invention are described above, the present invention is not limited to the embodiments described above but can be variously modified within the scope of the invention. In particular, the plurality of embodiments and variations described in this specification can be arbitrarily combined as necessary.

(A) Another Embodiment of Processing the Multiplexed Measurement Light Beam Received by the Light Detector

In the first to fourth embodiments, the analyzing unit 94, 4094 sets the second derivative value of the intensity of the multiplexed measurement light beam Lm (or the measurement light beam Lm′) received by the light detector 7, 405 as the reference intensity Is (or the first reference intensity Is1′, the second reference intensity Is2′) and the measured target intensity Id, Id′. However, the embodiments are limited to this. The numerical value data of the intensity of the multiplexed measurement light beam Lm (or the measurement light beam Lm′) received by the light detector 7, 405 without any processing may be set as the reference intensity and the measured target intensity. In this case, a light emission device other than the laser diode (for example, an LED or the like) may be used as the light source.

(B) Another Embodiment of the First Concentration in the Fourth Embodiment

In the fourth embodiment described above, the analyzing unit 4094 sets the first concentration C1 as a maximum concentration of the target gas. If the second reference intensity Is2′ including a clear peak due to the light absorption by the target gas can be obtained, the analyzing unit 4094 may set a relatively high, but not maximum, concentration of the target gas as the first concentration. In this case, the concentration of the target gas higher than the first concentration is calculated as a “negative concentration”.

(C) Another Embodiment of the Method of Analyzing the Target Gas in the Fourth Embodiment

In the fourth embodiment described above, after obtaining the second reference intensity Is2′, the analyzing unit 4094 performs the target gas analysis using only the analysis data generated by using the second reference intensity Is2′. Since the analysis data having a clear peak due to the light absorption may be used in order to accurately analyze the target gas, the analysis data is not limited to that calculated using the second reference intensity Is2′.

For example, the analysis data calculated using the first reference intensity Is1′ and the analysis data calculated using the second reference intensity Is2′ may be both obtained, then one of the two analysis data which includes a clearer peak due to the light absorption may be selected to perform the target gas analysis using the selected analysis data.

In this way, the target gas analysis can be accurately performed in a wider range of the concentration.

The present invention can be widely applied to the gas analyzing apparatus that analyzes target gases that possibly exist in the measurement space.

### LIST OF REFERENCE CHARACTERS

100, 200, 200′, 300, 400 Gas analyzing apparatus
1-1, 1-2, 1-3, 1-n, 401 Light source
11-1, 11-2, 11-n, 4011 Temperature controlling apparatus
3 Optical multiplexer

### 5 Inlet

50, 50′ light splitter
51 Optical fiber
51′-1, 51′-2, 51′-k, 51″-1, 51″-2 Optical outlet

### 53, 403, 53′-1, 53′-2, 53′-k Collimator

7, 405 Light detector
71, 407 Light collector
73 Light receiving device

### 9, 409 Controller

91, 4091 Light source controller

### 92, 4092 Storage

93, 4093 Data obtaining unit
94, 4094 Analyzing unit
95, 4095 Temperature controller
96 Gas type reporting unit

CH1 Process controller

CH, CH′, CH′-1, CH′-2, CH′-k Chamber

What is claimed is:

1. A gas analyzing apparatus analyzing gases existing in a measurement space, the apparatus comprising:
a plurality of light sources simultaneously outputting a plurality of measurement light beams of unique wavelength ranges, each of the plurality of measurement light beams capable of being absorbed by one of a plurality of target gases that exist in the measurement space;
an inlet introducing the plurality of measurement light beams output simultaneously from the plurality of light sources into the measurement space;
a light detector measuring a total intensity obtained by summing intensities of the plurality of measurement light beams that have been introduced from the inlet and have passed through the measurement space; and
an analyzing unit analyzing the plurality of target gases based on a difference between a measured target intensity and a reference intensity, the measured target intensity being the total intensity measured by the light detector when the plurality of measurement light beams pass through the measurement space in which one of the plurality of target gases exists, and the reference intensity being the total intensity measured by the light detector when the plurality of measurement light beams pass through the measurement space in which none of the plurality of target gases exists.

2. The gas analyzing apparatus according to claim 1, wherein
the inlet includes an optical multiplexer generating a multiplexed measurement light beam by multiplexing the plurality of measurement light beams output simultaneously from the plurality of light sources to introduce the multiplexed measurement light beam into the measurement space wherein the multiplexed measurement light beam has an intensity, and
the light detector measures intensity of the multiplexed measurement light beam as the total intensity.

3. The gas analyzing apparatus according to claim 2, wherein the inlet further includes a light splitter dividing the multiplexed measurement light beam so as to output the multiplexed measurement light beam from a plurality of light outlets.

4. The gas analyzing apparatus according to claim 3, wherein the inlet introduces each of the plurality of multiplexed measurement light beams output from the plurality of light outlets into each of a plurality of measurement spaces.

5. The gas analyzing apparatus according to claim 1, further comprising a gas type reporting unit reporting which target gas exists in the measurement space.

6. The gas analyzing apparatus according to claim 1, wherein each of the plurality of light sources increases or decreases the wavelength of the measurement light beam within the unique wavelength range at a predetermined period.

7. The gas analyzing apparatus according to claim 6, wherein a periodic increase/decrease pattern of some of drive currents input respectively to some of the plurality of light sources are different from periodic increase/decrease patterns of other drive currents.

8. The gas analyzing apparatus according to claim 6, wherein the analyzing unit analyzes the target gases based on a difference between a temporal change of the measured target intensity and a temporal change of the reference intensity.

9. A gas analyzing apparatus analyzing a target gas existing in a measurement space, the apparatus comprising:
a light source outputting a measurement light beam having a unique wavelength range, the measurement light beam capable of being absorbed by the target gas;
an inlet introducing the measurement light beam output from the light source into the measurement space;
a light detector measuring an intensity of the measurement light beam that has been introduced from the inlet and passed through the measurement space; and
an analyzing unit analyzing the target gas in the measurement space in which the target gas exists at a concentration lower than a first concentration, based on a difference between a reference intensity and a measured target intensity, the reference intensity being the intensity measured by the light detector when the measurement light beam passes through the measurement space in which the target gas exists at the first concentration, and the measured target intensity being the intensity measured by the light detector when the measurement light beam passes through the measurement space in which the target gas exists at a concentration lower than the first concentration.

10. The gas analyzing apparatus according to claim 9, wherein the light source is configured to increases or decreases the wavelength of the measurement light beam within the unique wavelength range at a predetermined period.

11. The gas analyzing apparatus according to claim 10, wherein the analyzing unit analyzes the target gas based on a difference between a temporal change of the measured target intensity and a temporal change of the reference intensity.
